(19) United States
(12) Patent Application Publication
(10) Pub. No.: US 2010/0179209 A1
(43) Pub. Date: Jul. 15, 2010

(54) Pactimibe medicaments for preventing/treating a disease due to sebaceous gland dysfunction in humans or animals

(75) Inventor: Thibaud BIADATTI-PORTAL, Opio (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/688,310

(22) Filed: Jan. 15, 2010

Related U.S. Application Data
(63) Continuation of application No. PCT/FR2008/051345, filed on Jul. 17, 2008.

(30) Foreign Application Priority Data
Jul. 19, 2007 (FR) 07/56602

(51) Int. Cl. A61K 31/404; A61P 17/10

(52) U.S. Cl. 514/415

(57) ABSTRACT

Administration of at least one compound selected from N-(1-octyl-5-carboxymethyl-4,6-dimethylindolin-7-yl)-2,2-dimethylpropanamide (pactimibe) of formula (I):

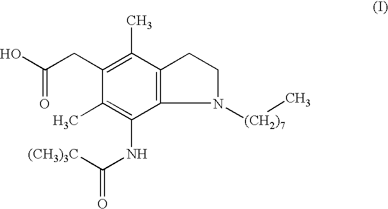

or pharmaceutically acceptable salt and pharmaceutically acceptable solvate thereof, is useful for preventing or treating a disorder due to a sebaceous gland dysfunction in humans or animals, for example acne and/or any state or pathological condition related to an overproduction of sebum, e.g., seborrhoeic dermatitis, greasy skin or a greasy scalp.

### CROSS-REFERENCE TO PRIORITY/PCT APPLICATIONS

This application claims priority under 35 U.S.C. §119 of FR 07/56602, filed Jul. 19, 2007, and is a continuation/national phase of PCT/FR 2008/051345, filed Jul. 17, 2008 and designating the United States (published in the French language on Jan. 29, 2009 as WO 2009/013433 A2; the title and abstract were also published in English), each hereby expressly incorporated by reference in its entirety and each assigned to the assignee hereof.

### BACKGROUND OF THE INVENTION

1. Technical Field of the Invention

The present invention relates to the formulation of pactimibe, or of a pharmaceutically acceptable salt or pharmaceutically acceptable solvate thereof, into medicaments for preventing or treating a disorder due to a sebaceous gland dysfunction in humans or animals, for instance acne and/or any state or pathological condition related to an overproduction of sebum, e.g., dermatitis, greasy skin or a greasy scalp.

2. Description of Background and/or Related and/or Prior Art

EP-0866059 describes a family of heterocyclic derivatives which are inhibitors of the ACAT (acyl CoA: cholesterol O-acyltransferase) enzyme. These compounds are described herein as compounds which can be used for the treatment of arteriosclerosis, hyperlipidaemia, in diabetes and cerebrovascular and cardiovascular ischaemic diseases. Inhibition of the ACAT enzyme blocks the esterification of free cholesterol to give cholesterol esters. Moreover, this enzyme is expressed in the sebaceous glands. The sebaceous glands are holocrine glands which secrete a mixture of lipids that is known as sebum. Cholesterol esters make up 2-3% of the lipids of human sebum. Excess sebum promotes the appearance of acne or of other dermatological pathological conditions, and in any event, gives the skin a greasy and relatively unattractive appearance. Current treatments for decreasing excess sebum are unsatisfactory. By way of example, representative is administration of isotretinoin which, although it decreases sebum production by close to 90%, has considerable side effects. No effective treatment based on an ACAT enzyme inhibitor has to date been developed.

### SUMMARY OF THE INVENTION

It has now been demonstrated that, among the ACAT enzyme inhibitors described in EP-0866059, N-(1-octyl-5-carboxymethyl-4,6-dimethylindolin-7-yl)-2,2-dimethylpropanamide, known as pactimibe, of formula (I):

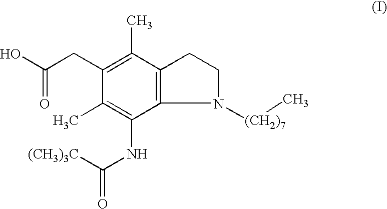

optionally in the form of a pharmaceutically acceptable salt or solvate, thereof, is useful for preventing or treating disorders due to a sebaceous gland dysfunction in humans or animals and/or any state or pathological condition related to an overproduction of sebum.

Therefore, the present invention features formulation of at least one compound selected from among N-(1-octyl-5-carboxymethyl-4,6-dimethylindolin-7-yl)-2,2-dimethylpropanamide (pactimibe) of formula (I):

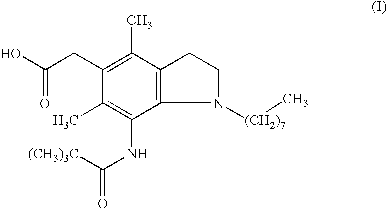

and pharmaceutically acceptable salts and pharmaceutically acceptable solvates thereof, into medicaments for preventing or treating a disorder due to a sebaceous gland dysfunction in humans or animals, and/or any state or pathological condition related to an overproduction of sebum.

One aspect of this invention is a regime or regimen comprising administering pactimibe, pharmaceutically acceptable salts and pharmaceutically acceptable solvates thereof, for the treatment or prevention of a disorder due to a sebaceous gland dysfunction in humans or animals and/or any state or pathological condition related to an overproduction of sebum.

### DETAILED DESCRIPTION OF BEST MODE AND SPECIFIC/PREFERRED EMBODIMENTS OF THE INVENTION

Preferably, administration of pactimibe, pharmaceutically acceptable salts and pharmaceutically acceptable solvates thereof, is useful for the treatment or prevention of acne, seborrhoeic dermatitis, greasy skin or a greasy scalp.

Pactimibe is described in Example 4 of EP-0866059. The pharmaceutically acceptable salts of the compound of formula (I) comprise those with inorganic or organic acids, such as hydrochloric acid, hydrobromic acid, sulfuric acid, phosphoric acid, nitric acid, picric acid, oxalic acid, sulfonic acids such as methanesulfonic acid, benzenesulfonic acid, p-toluenesulfonic acid, mesitylenesulfonic acid or naphthalenesulfonic acid, tartaric acid, dibenzoyltartaric acid, mandelic acid, acetic acid, propionic acid, lactic acid, citric acid, succinic acid, fumaric acid, maleic acid, malonic acid, malic acid, phthalic acid or camphorsulfonic acid. The salts of the compounds of formula (I) also comprise salts with organic or inorganic bases, for example the salts of alkali or alkaline-earth metals, such as the sodium, lithium, potassium, calcium or magnesium salts, or the salts with an amine, such as trometamol, or else the arginine or lysine salts or the salts of any physiologically acceptable amine, such as trimethylamine, triethylamine, tributylamine, pyridine, N-methylpiperidine, N-methylmorpholine, diethylamine, cyclohexylamine, procaine, dibenzylamine, N-benzyl-β-phenethylamine, 1-ephenamine or N,N′-dibenzylethylenediamine. Preferably, the pactimibe hydrochloride, the pactimibe sulfate, the pactimibe nitrate and the sodium salt of pactimibe, respectively described in Examples 5, 6, 7 and 8 of EP-0866059 are administered according to the invention.

The solvates of pactimibe or of salts thereof represent the hydrates of such compounds and/or the combination of such compounds with a linear or branched C₁-C₄ alcohol such as methanol, ethanol, isopropanol or n-propanol.

According to the present invention, it has been demonstrated that pactimibe is useful to combat disorders due to a sebaceous gland dysfunction in humans or animals and/or any state or pathological condition related to an overproduction of sebum.

The expression “disorders due to a sebaceous gland dysfunction” is preferably intended to mean acne.

The expression “state or pathological condition related to an overproduction of sebum” means in particular seborrhoeic dermatitis, greasy skin or a greasy scalp.

When it comprises a medicament, pactimibe, or a pharmaceutically acceptable salt or a pharmaceutically acceptable solvate thereof, is formulated as a pharmaceutical, and preferably dermatological, composition.

The present invention therefore also features pharmaceutical, preferably dermatological, compositions comprising, formulated into a physiologically acceptable carrier, at least one compound selected from among pactimibe, pharmaceutically acceptable salts and pharmaceutically acceptable solvates thereof, useful for the treatment or prevention of disorders due to a sebaceous gland dysfunction and/or any state or pathological condition related to an overproduction of sebum.

The term “physiologically acceptable carrier” means a medium that is compatible with the skin, the mucous membranes and/or the skin appendages.

Such compositions are therefore suitable for oral, topical, enteral, parenteral, ocular, sublingual, inhaled, subcutaneous, intramuscular, intravenous, transdermal, local or rectal administration. The oral and topical forms are nevertheless preferred. Pactimibe, optionally in the form of a pharmaceutically acceptable salt, or solvate, alone or in combination with another active ingredient, may be administered in a unit administration form, as a mixture with conventional pharmaceutical carriers or excipients, to animals and to human beings. Preferably, the pharmaceutical composition is packaged in a form suitable for oral or topical application. The term “topical application” means application to the skin and/or the mucous membranes.

The compositions according to the invention contain pactimibe, or a pharmaceutically acceptable form thereof, or pharmaceutically acceptable solvates thereof, in sufficient amount to obtain the desired prophylactic or therapeutic effect. The useful dosage varies according to the age, gender and weight of the patient. The pactimibe or a salt thereof, or solvates thereof, will preferably be administered in a proportion of from 0.01 to 100 mg/kg per day, advantageously of from 0.01 to 50 mg/kg per day. It is also possible to administer such doses in 2 to 4 daily administrations. Although these dosages are examples of average situations, there may be particular cases where higher or lower dosages are appropriate; such dosages are also according to the invention.

The compositions according to the invention comprise a physiologically acceptable carrier or at least one pharmaceutically acceptable excipient, selected according to the pharmaceutical form desired and the method of administration selected. Said carrier comprises at least one pharmaceutically acceptable excipient.

For oral administration, the pharmaceutical, preferably dermatological, composition may be in the form of tablets, gel capsules, sugar-coated tablets, pills, syrups, suspensions, solutions, powders, granules, emulsions, capsules or microspheres or nanospheres or lipid of polymeric vesicules for controlled release. For parenteral administration, the composition may be in the form of solutions or suspensions for infusion or for injection. To obtain a solid composition for oral administration, the active ingredient may be mixed with at least one inert diluent, such as sucrose, lactose or starch. In general, other additives, such as a lubricant, for instance magnesium stearate, may be added. In the case of capsules, tablets or pills in particular, a buffer may be added. In the case of oral liquid compositions, an inert diluent such as water may be used.

When applied topically, the pharmaceutical composition according to the invention is more particularly useful for the treatment of the skin and the mucous membranes and may be in the form of salves, creams, milks, ointments, powders, impregnated pads, syndets, solutions, gels, sprays, foams, suspensions, lotions, sticks, shampoos or washing bases. It may also be in the form of suspensions of microspheres or nanospheres or lipid or polymeric vesicules or of polymeric patches and hydrogels for controlled release. This composition for topical application may be in anhydrous form, in aqueous form or in the form of an emulsion.

When pactimibe (I), or a salt or solvate thereof, is administered orally, it is administered in a proportion of from 0.01 to 100 mg/kg per day, advantageously from 0.01 to 50 mg/kg per day.

When pactimibe (I), or a salt or solvate thereof, is administered topically, it is used at a concentration of generally from 0.001% and 10% by weight, preferably from 0.01% and 5% by weight, relative to the total weight of the composition.

For the treatment of disorders due to sebaceous gland dysfunctions, pactimibe, or a pharmaceutically acceptable salt or a pharmaceutically acceptable solvate thereof, may also be administered in combination with another active ingredient.

The pharmaceutical, preferably dermatological, compositions as described above may therefore contain inert additives, or even pharmacodynamically active additives, or combinations of these additives, and in particular:

wetting agents;

flavor enhancers;

preservatives such as para-hydroxybenzoic acid esters;

stabilizers;

moisture regulators;

pH regulators;

osmotic pressure modifiers;

emulsifiers;

UV-A and UV-B screening agents;

emollients;

moisturizers such as glycerol, PEG 400, thiamorpholinone, and its derivatives, or urea.

Of course, one skilled in the art will take care to select the optional compound(s) to be added to these compositions in such a way that the desired effect on disorders due to sebaceous gland dysfunctions is not, or not substantially, impaired by the envisaged addition.

To further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Compositions

A—Oral Administration:

0.2 g tablet:

| | |
|---|---|
| Pactimibe | 0.001 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

B—Topical Administration:

(a) Salve

| | |
|---|---|
| Pactimibe | 0.30 g |
| Codex white petroleum jelly | qs 100 |

(b) Lotion

| | |
|---|---|
| Pactimibe | 0.10 g |
| Polyethylene glycol (PEG 400) | 69.90 g |
| 95% ethanol | 30.00 g |

The study of the properties of pactimibe showed that pactimibe, and also the pharmaceutically acceptable salts or solvates thereof, are not toxic and make it possible to reduce sebum production by the sebaceous glands.

### Example 2

### Biological Results

Measurement of ACAT/SOAT Activity:

The objective of this test is to visualize the activity of the ACAT/SOAT enzyme on the synthesis of cholesterol esters.

The test is based on the following publication: “Identification of ACAT1- and ACAT2-specific inhibitors using a novel, cell based fluorescence assay: individual ACAT uniqueness”, J. Lipid Res., (2004) vol. 45, pages 378-386.

The principle of this test is based on the use of NBD-cholesterol, which is a cholesterol analogue of which the fluorescence is dependent on its environment. When said analogue is in a polar environment, it is weakly fluorescent, whereas in a non-polar environment, it is strongly fluorescent. Free NBD-cholesterol is localized in the cell membranes and is weakly fluorescent in this polar environment. When NBD-cholesterol is esterified by ACAT, the NBD-cholesterol ester is localized in non-polar lipid droplets and is therefore strongly fluorescent.

Method:

HepG2 cells are incubated in the presence of NBD-cholesterol (1 μg/ml) and of the test product in transparent-bottomed black 96-well plates at a rate of 30 000 cells per well. After incubation for 6 h at 37° C., 5% CO₂, the medium is removed by turning the plates over and the cells are washed with twice 100 μl of PBS. After the addition of 50 μl of lysis buffer (10 mM NaPO₄, 1% Igepal), the plates are shaken for 5 min and read by fluorescence (excitation 490 nm, emission 540 nm) on the Fusion instrument (Perkin Elmer).

In this test, pactimibe strongly inhibits cholesterol ester synthesis with an IC₅₀ of 50 nM.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A pharmaceutical/dermatological composition useful for preventing or treating a disorder due to a sebaceous gland dysfunction in humans or animals and/or any state or pathological condition related to an overproduction of sebum comprising a thus effective amount of N-(1-octyl-5-carboxymethyl-4,6-dimethylindolin-7-yl)-2,2-dimethylpropanamide (pactimibe) of formula (I): 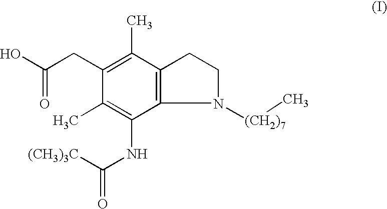
or pharmaceutically acceptable salt or pharmaceutically acceptable solvate thereof, formulated into a physiologically acceptable carrier therefor.

2. The pharmaceutical/dermatological composition as defined by claim 1, formulated for oral administration.

3. The pharmaceutical/dermatological composition as defined by claim 1, formulated for topical administration.

4. The pharmaceutical/dermatological composition as defined by claim 1, formulated in the form of a salve, cream, milk, ointment, powder, impregnated pads, syndet, solution, gel, spray, foam, suspension, lotion, stick, shampoo or washing base.

5. A regime or regimen for preventing or treating a disorder due to a sebaceous gland dysfunction in humans or animals and/or any state or pathological condition related to an overproduction of sebum, comprising administering to a human or animal in need of such treatment, a thus effective amount of a pharmaceutical/dermatological composition as defined by claim 1.

6. The regime or regimen as defined by claim 5, comprising the treatment of acne.

7. The regime or regimen as defined by claim 5, comprising the treatment of seborrhoeic dermatitis, greasy skin or a greasy scalp.

8. The pharmaceutical/dermatological composition as defined by claim 1, comprising pactimibe nitrate, pactimibe sulfate, pactimibe hydrochloride and/or the sodium salt of pactimibe.
